(19) United States
(12) Patent Application Publication
(10) Pub. No.: US 2005/0171544 A1
(43) Pub. Date: Aug. 4, 2005

(54) Bone plate with toothed aperture

(75) Inventor: James G. Falkner JR., Hillsboro, OR (US)

Correspondence Address: KOLISCH HARTWELL, P.C., 520 S.W. YAMHILL STREET, SUITE 200, PORTLAND, OR, 97204, US

(73) Assignee: Acumed LLC

(21) Appl. No.: 11/050,342

(22) Filed: Feb. 2, 2005

Related U.S. Application Data
(60) Provisional application No. 60/541,414, filed on Feb. 2, 2004.

(51) Int. Cl. A61B 17/58

(52) U.S. Cl. 606/69

(57) ABSTRACT

Systems, including methods and apparatus, and kits, for fixing bones using bone plates having toothed apertures for retaining fasteners.

### CROSS-REFERENCE TO PRIORITY APPLICATION

This application is based upon and claims the benefit under 35 U.S.C. § 119(e) of U.S. Provisional Patent Application Ser. No. 60/541,414, filed Feb. 2, 2004, which is incorporated herein by reference in its entirety for all purposes.

### INTRODUCTION

The human skeleton is composed of 206 individual bones that perform a variety of important functions, including support, movement, protection, storage of minerals, and formation of blood cells. To ensure that the skeleton retains its ability to perform these functions, and to reduce pain and disfigurement, bones that become damaged should be repaired promptly and properly. Typically, a fractured or cut bone is treated using a fixation device, which reinforces the bone and keeps it aligned during healing. Fixation devices may include external fixation devices (such as casts and fixators) and/or internal fixation devices (such as bone plates, nails, and/or bone screws), among others.

Bone plates are sturdy internal devices, usually made of metal, that mount directly to the bone adjacent a fracture (or other bone discontinuity). To use a bone plate to repair a discontinuity of a bone, a surgeon typically (1) selects an appropriate plate, (2) reduces the discontinuity (e.g., sets the fracture), and (3) fastens the plate to bone fragments disposed on opposite sides of the discontinuity using suitable fasteners, such as screws and/or wires, so that the bone plate spans the discontinuity and the bone fragments are fixed in position.

Fixation of bone fractures (or other discontinuities) can be problematic when these fractures are disposed near the ends of bones (“metaphyseal fractures”). Standard bone plates secured only to the exterior surface of the fractured bones may not be sufficient to immobilize metaphyseal fragments. In particular, these fragments may have lower bone density and/or quality so that bone screws cannot achieve sufficient purchase in bone. Accordingly, metaphyseal fractures may not heal properly during fixation, resulting in malunion or persistent nonunion at the fracture sites. Problems with metaphyseal fractures that do not heal properly can be particularly prevalent in larger bones that support greater loads, such as the humerus, the tibia, and the femur, among others.

Blade plates may provide a suitable alternative to standard bone plates for fixation of metaphyseal fractures. Blade plates may be shaped to define two adjacent plate portions: an anchor portion, and a blade portion extending transverse to the anchor portion. The anchor portion may be configured, as in a standard plate, to be secured to an exterior surface of a fractured bone, generally extending to a more central (diaphyseal) region of the bone. In contrast, the blade portion may be placed in the interior of the bone, generally in the end (metaphyseal) region of the bone, so that the anchor and blade portions are disposed on opposite sides of the fracture. The blade portion, when properly secured to an end region, thus may stabilize the end region against movement relative to the central region of the bone, to promote healing of the fracture.

An “interlocking” bone screw has been used to secure the blade portion, when inside bone, to an end region of a fractured bone. The interlocking screw may span the anchor and blade portions (and the fracture) to “interlock” and tension these portions. For example, the interlocking screw may be placed first through an opening of the anchor portion, next through bone and across the fracture, and then through an opening in the blade portion. The interlocking screw may be oversized, to engage the blade portion in an interference fit at the opening of the blade portion and to limit movement of the blade portion relative to the interlocking screw. However, the interference fit may be difficult to achieve reproducibly and may not be tight enough to prevent slippage of the blade portion relative to the interlocking screw.

### SUMMARY

The present teachings provide systems, including methods, apparatus, and kits, for fixing bones using bone plates having toothed apertures for retaining fasteners.

### BRIEF DESCRIPTION OF THE DRAWINGS

FIG. 1 is a sectional view of an exemplary system for fixing bones using a bone plate with a toothed aperture, with the bone plate secured to a fractured bone, in accordance with aspects of the present teachings.

FIG. 2 is a perspective view of the bone plate of FIG. 1, in accordance with aspects of the present teachings.

FIG. 3 is a sectional view of the bone plate of FIG. 1 illustrating exemplary alternative axes along which a bone screw may be directed into threaded engagement with the toothed aperture, in accordance with aspects of the present teachings.

FIG. 4 is a fragmentary sectional view of selected portions of the bone plate of FIG. 1, particularly a bridge portion and an internal portion of the bone plate including the toothed aperture, in accordance with aspects of the present teachings.

FIG. 5 is a view of the bone plate of FIG. 4, taken generally along line 5-5 of FIG. 4.

FIG. 6 is a view of the bone plate and bone screw of FIG. 3, taken generally along line 6-6 of FIG. 3.

FIG. 7 is view of the bone plate of FIG. 6, taken as in FIG. 6 but with the bone screw removed.

FIG. 8 is a sectional view of another exemplary bone plate with a toothed aperture, showing a drill guide in threaded engagement with an opening of the bone plate and defining a guide axis that extends through the toothed aperture, in accordance with aspects of the present teachings.

FIG. 9 is a sectional view of yet another exemplary bone plate with a toothed aperture, with a plurality of threaded fasteners extending from plate openings into threaded engagement with the toothed aperture, in accordance with aspects of the present teachings.

FIG. 10 is a fragmentary sectional view of selected portions of an exemplary bone plate having a toothed aperture with nonparallel ridges, in accordance with aspects of the present teachings.

FIG. 11 is a fragmentary sectional view of selected portions of another exemplary bone plate having a toothed aperture with nonparallel ridges, in accordance with aspects of the present teachings.

### DETAILED DESCRIPTION

The present teachings provide systems, including methods, apparatus, and kits, for fixing bones using bone plates having toothed apertures for retaining fasteners. The bone plates may include first and second plate portions disposed adjacent one another. Each plate portion may be an exterior portion configured to be apposed and secured to an exterior surface of bone. Alternatively, one or more of the plate portions may be an interior portion configured to be disposed interior to bone, as in a blade plate. Each bone plate portion may define one or more openings for receiving fasteners, such as bone screws, that secure the plate portions to bone.

At least one of the openings may be a toothed aperture, that is, an opening having one or more projections or “teeth” at its perimeter. The toothed aperture may be configured to receive a threaded shank of a bone fastener and retain the threaded shank by rotational coupling with one or more teeth of the aperture, to achieve threaded engagement between the threaded shank and the teeth. Accordingly, the toothed aperture may include teeth configured as relatively short thread-like segments, possibly but not necessarily helical in structure. In some examples, the toothed aperture may be configured to receive and retain one or more bone fasteners that extend from one or more openings of the plate, for intra-plate placement of bone fasteners. Bone plates having toothed apertures may provide, among others, (1) more options for placement of bone screws (more screws per aperture and/or a greater range of permissible screw angles, (2) increased tolerance for misguided screws, (3) improved retention of bone screws, (4) a greater range of permissible angles to which the plate can be bent, and/or (5) intra-plate tensioning, for example, to compress bone.

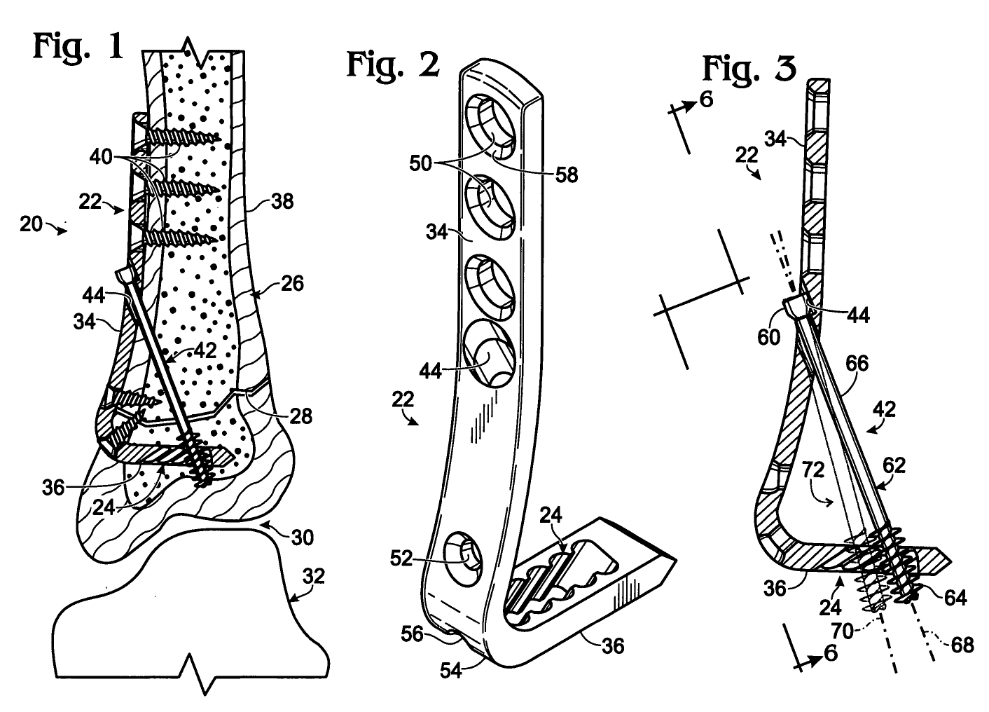

FIG. 1 shows an exemplary system 20 for fixing bones using a bone plate 22 with a toothed aperture 24. The bone plate may be positioned on and/or in any suitable bone(s) to span any natural or artificial discontinuity within a bone or between bones. In the present illustration, plate 22 is secured to a distal end (metaphyseal) region of a tibia bone 26 and spans fracture 28. In other examples, plate 22 may span a joint, such as joint 30 between tibia 26 and talus 32, among others.

Bone plate 22 may have first and second plate portions 34, 36 configured to be disposed in any suitable positions relative to the bone. In the present illustration, the first and second plate portions 34, 36 are disposed so that they are, respectively, external to (on) and internal to (in) tibia 26.

First (external) plate portion 34 may be secured to tibia 26 adjacent diaphyseal region 38 of the bone using any suitable bone fasteners, such as bone screws 40 in the present illustration. The bone screws may be placed into bone from any suitable number of openings of the bone plate. The external plate portion may be contoured to follow an exterior surface of the bone.

Second (internal) plate portion 36 may be disposed substantially interior to tibia 26 (or in a bone adjacent to the tibia) and/or may be apposed to surfaces of the bone that normally lie within the bone. The internal plate portion may define toothed aperture 24, which is configured to receive and retain a threaded fastener 42. The threaded fastener may extend into and/or through only one opening (a toothed aperture) or at least two openings of the bone plate, such as an opening 44 in the external plate portion. Alternatively, or in addition, the threaded fastener may extend between two or more distinct plates.

Further aspects of the present teachings are described in the following sections, including (I) overview of bone plates, (II) plate portions, (III) toothed apertures, (IV) methods of using bone plates with toothed apertures, and (V) examples.

I. Overview of Bone Plates

Bone plates as described herein generally comprise any relatively low-profile (or plate-like) fixation device configured to stabilize at least one bone by attachment to the bone. The fixation device may be configured to span any suitable bone discontinuity so that the fixation device fixes the relative positions of bone fragments (and/or bones) disposed on opposing sides of the bone discontinuity.

Suitable discontinuities may occur naturally and/or may result from injury, disease, and/or surgical intervention, among others. Accordingly, exemplary discontinuities for use with the bone plates described herein may include joints, fractures (breaks in bones), osteotomies (cuts in bones), and/or nonunions (for example, produced by injury, disease, or a birth defect), among others.

The bone plates described herein may be configured for use on any suitable bone of the human skeleton and/or of another vertebrate species. Exemplary bones may include bones of the arms (radius, ulna, humerus), legs (femur, tibia, fibula, patella), hands/wrists, feet, vertebrae, scapulas, pelvic bones, cranial bones, ribs, and and/or clavicles, among others. Particular examples where bone plates having toothed apertures may be suitable include, but are not limited to, discontinuities in or adjacent metaphyseal regions of long bones, such as proximal or distal regions of the humerus, the tibia, and/or the femur. Alternatively, or in addition, these bone plates may be used to fuse joints, such as fusion of the tibiotalocalcaneal (ankle) joint, among others.

Each bone plate may be configured to be disposed in any suitable position relative to its target bone. The bone plate (or a plate portion, see Section II) may be configured to be disposed in contact with an exterior surface of the bone and thus may be positioned at least substantially (or completely) exterior to the bone. Alternatively, the bone plate may be configured to be disposed at least partially interior to a bone, that is, apposed to (normally) interior bone surfaces (i.e., subchondral bone) when secured to the bone. The interior bone surfaces may be created during installation of the bone plate (such as by punching the bone plate through the exterior bone surface) and/or may be accessible due to a break, a cut, and/or the like.

The bone plates may be formed of any suitable materials. The bone plates may be of a sturdy yet malleable construction. Generally, the bone plates should be stiffer and stronger than the section of bone spanned by the plates, yet flexible (e.g., springy) enough not to strain the bone significantly. Suitable materials for forming the bone plates may be biocompatible materials (such as titanium or titanium alloys, cobalt chromium, stainless steel, plastic, ceramic, etc.) and/or bioabsorbable materials (such as polygalactic acid (PGA), polylactic acid (PLA), copolymers thereof, etc.), among others.

The bone plates may be configured to reduce irritation to the bone and surrounding tissue. For example, the bone plates may be formed of a biocompatible material, as described above. In addition, the bone plates may have a low and/or feathered profile to reduce their protrusion into adjacent tissue and rounded, burr-free surfaces/edges to reduce the effects of such protrusion.

The bone plates described herein may be sized and shaped to conform to particular portions of a bone (or bones). The plates may be generally elongate, with a length L, a width W, and a thickness T. Here, length L≧width W≧thickness T. In use, the long axis of the bone plates (or of a plate portion) may be aligned with the long axis of the corresponding bone or may extend obliquely or even transversely relative to the bone's long axis. The length and/or width of the bone plates may be varied according to the intended use, for example, to match the plates with a preselected region of bone(s) and/or a particular injury to the bone. For example, the plates may be generally linear for use on the shaft of a long bone and/or may have a nonlinear shape, such as for use near an end of a bone. In some embodiments, the bone plates may be configured for use on both sides of the body, such as when the bone plates are bilaterally symmetrical. In some embodiments, the bone plates may be asymmetrical and configured for use on either the left or the right side of the body.

The bone plates (or exterior plate portions, see Section II) may include inner (bone-facing) and outer (bone-opposing) surfaces. One or both of these surfaces may be contoured generally to follow an exterior surface of a target bone (or bones) for which a bone plate is intended, so that the bone plate maintains a low profile and fits onto the bone(s). For example, the inner surface of a plate (or of an exterior plate portion) may be generally complementary in contour to the bone surface. The outer surface of the plate (or of an exterior plate portion) also may correspond in contour to the bone surface and may be generally complementary to the inner surface of the plate. The bone plates may be partially and/or completely precontoured, at the time of manufacture, allowing physicians to apply them to bone(s) with little or no additional bending at the time of application.

The thickness of the bone plates may be defined by the distance between the inner and outer surfaces of the plates. The thickness of the plates may vary between plates and/or within the plates, according to the intended use. For example, thinner plates may be configured for use on a smaller bone and/or on a bone or bone region where soft tissue irritation is a greater concern. Thickness may be varied within the plates. For example, the plates may become thinner as they extend over protrusions (such as processes, condyles, tuberosities, and/or the like), reducing their profile and/or rigidity, among others. Alternatively, or in addition, the thickness may vary as an internal portion of the bone plate extends into bone, for example, becoming thinner to facilitate insertion of this internal portion or thicker to increase structural stability. The thickness of the plates also may be varied to facilitate use, for example, to make the plates thinner where they typically need to be deformed by bending and/or twisting the plates, such as at the junction (or bridge region) between plate portions (see Section II). In this way, the plates may be thicker and thus stronger in regions where they may not need to be contoured, such as along the shaft of the bone.

The bone plates generally include a plurality of openings, also termed apertures. The openings may be adapted to receive fasteners for securing the plates to bone. Alternatively, or in addition, the openings may be adapted (1) to alter the local rigidity of the plates, (2) to permit the plates to be manipulated with a tool (such as an attachable handle), (3) to facilitate attachment of a guide for drilling and/fastener placement, and/or (4) to facilitate blood flow to bone regions where the bone plates are installed, to promote healing, among others.

The openings may have any suitable positions, sizes, and/or densities within each portion of a bone plate. The openings may be arrayed generally in a line along a portion of the plate, for example, centered across the width of the plate. Alternatively, the openings may be arranged nonlinearly, for example, disposed in a staggered arrangement. In some embodiments, the openings may be configured so that a set of bone screws can be directed along nonparallel paths, for example, to increase the purchase of the set of bone screws on bone. Further aspects of openings configured to direct bone screws, particularly unicortical bone screws, along nonparallel paths are included in the following patent application, which is incorporated herein by reference: U.S. patent application Ser. No. 10/968,850, filed Oct. 18, 2004.

The openings may have any suitable shape and structure. Exemplary shapes may include circular, elliptical, rectangular, elongate, etc. The openings may include counterbores configured, for example, to receive a head of a bone screw. The openings may be threaded or nonthreaded, and each bone plate may include one or more threaded and/or nonthreaded openings. In some embodiments, the plates may include one or a plurality of elongate openings (slots) extending axially, obliquely, and/or transversely along each bone plate. The elongate openings may be compression slots that include counterbores to provide compression when heads of bone screws are advanced against the counterbores. Alternatively, or in addition, the elongate openings may be used to adjust the position of bone plates and/or plate portions relative to bone before the plates are fully secured to the bone. One or more of the elongate openings of each bone plate may be a toothed aperture (see Sections III and V).

The fasteners generally comprise any mechanism for affixing a bone plate to a bone, including screws, pins, and/or wires, among others. A preferred fastener is a bone screw, including unicortical, bicortical, and/or cancellous bone screws. Unicortical and bicortical bone screws typically have relatively small threads for use in hard bone, such as typically found in the shaft portion of a bone, whereas cancellous bone screws typically have relatively larger threads for use in soft bone, such as typically found near the ends (metaphyseal regions) of a long bone. Unicortical bone screws penetrate the bone cortex once, adjacent the bone plate. Bicortical bone screws penetrate the bone cortex twice, adjacent the bone plate and opposite the bone plate. Generally, unicortical screws provide less support than bicortical screws, because they penetrate less cortex. The size and shape of the fasteners may be selected based on the size, shape, and disposition of the openings, or vice versa. For example, unicortical bone screws may be suitable with particular arrangements of openings, as described above. In some examples, the bone screws may include a shaft that includes a distal threaded region and a proximal nonthreaded region. This arrangement of threaded and nonthreaded regions may permit the screw to function as a compression screw that spans plate portions and applies an adjustable tension between the plate portions.

In some examples, the bone plates may include projections, such as prongs, configured to be received in bone to restrict movement of the bone plates. The projections may be sharp or blunt according to their intended use. Prongs may be used in place of, or in addition to, bone fasteners, for one or more portions of each bone plate.

II. Plate Portions

The bone plates may have at least one, and generally two or more, plate portions (or anchor portions) configured to be secured to different regions of a bone (or bones). Each plate portion may be structured for a specific region of a bone. For example, the bone plates may include a proximal plate portion for attachment to a more proximal region of a bone, and a distal plate portion for attachment to a more distal region of the same bone. The bone plates may include an external plate portion configured to fit against an exterior surface region of bone adjacent a bone discontinuity. Alternatively, or in addition, the bone plates may include an internal plate portion configured to be received in an interior region of bone (generally, subchondral bone) adjacent the bone discontinuity.

The plate portions of a bone plate may have any suitable connection. In some examples, two or more of the plate portions (or the entire bone plate) may be formed integrally, so that a unitary bone plate includes the plate portions. Alternatively, plate portions may be formed as separate pieces. The separate pieces may be connected by any suitable connection and/or joint, including a fastener(s), welding, a hinge joint, a ball in socket, and/or the like. Further aspects of bone plates having adjustable joints are described in the following patent application, which is incorporated herein by reference: U.S. patent application Ser. No. 10/716,719, filed Nov. 19, 2003. In some examples, the bone plate may be two separate bone plate components configured to be connected by one or more fasteners, such as fasteners that extend from one of the plate components, through bone, and into engagement with another of the plate components.

The plate portions of a bone plate may have any suitable relative disposition. The plate portions may be disposed substantially collinear and/or parallel, oblique, or substantially transverse to one another. In some examples, the plate portions may define planes that are disposed oblique or substantially transverse to each other. For example, the bone plate may have an exterior plate portion that fits against the exterior surface of a bone and an interior plate portion that extends at least substantially transverse to the exterior surface to be received in the interior of the bone. Accordingly, in some examples, the bone plate may be generally L-shaped, such as when viewed in profile.

The relative disposition of plate portions may be fixed and/or adjustable. In some examples, the plate portions may be connected integrally by a deformable bridge region, so that the bone plate can be bent pre- or peri-operatively to adjust the relative disposition of the plate portions. Alternatively, the plate portions may be distinct pieces connected, for example, through an adjustable joint, as described above.

Each plate portion may have one or more openings. Each opening may be configured to receive a bone fastener for placement of the bone fastener into bone. Alternatively, or in addition, the opening may be configured to enable the fastener to span plate portions by extending between an opening in each of two or more plate portions, such as a first opening in a first plate portion and a toothed aperture in a second plate portion.

The first opening may have any suitable configuration. For example, the first opening may be configured to receive a head of the fastener and the toothed aperture may be configured to receive a shank of the fastener, or vice versa. The first opening may be threaded or nonthreaded, and may be circular or elongate. Accordingly, the first opening may restrict a fastener of a suitable diameter to substantially one direction of approach (such as with a circular first opening and a close-fitting fastener), or the first opening may be an elongate opening (or, for example, a circular opening large enough to provide a loose fit with the fastener) that permits this fastener be placed at a disposition selected from a range of angular dispositions. In any case, the first opening may define an axis that intersects the toothed aperture suitable for threaded engagement of a fastener. In some examples, the first plate portion may include two or more first openings that permit two or more fasteners to be disposed, alternately and/or concurrently, in threaded engagement with the toothed aperture.

An internal plate portion may be configured for installation into bone. Accordingly, the internal plate portion may be thinner than the external plate portion. Alternatively, or in addition, the internal plate portion may taper toward an end of the plate, to provide a leading edge that enters bone first, so that the leading edge can penetrate bone more easily.

The internal plate portion may have fewer openings than the external plate portion. In some embodiments, the internal plate portion may have one or more toothed apertures and no additional openings. Alternatively, the internal plate portion may have one or more additional (toothed or nontoothed) openings.

III. Toothed Apertures

Each bone plate may include one or more toothed apertures. A toothed aperture, as used herein, is any opening (aperture) having one or more projections (teeth) that adjoin the opening. The projections may be formed by any suitable portion of the bone plate, and generally by a wall that defines at least a portion of the perimeter of the aperture. The projections may be configured so that the toothed aperture can receive and axially retain a shank (and/or other feature), particularly a threaded shank, of a fastener.

The toothed aperture may have any suitable shape and position in the bone plate. In some examples, the toothed aperture may be elongate, such as generally oval or rectangular, among others. The toothed aperture may extend generally parallel, oblique, or transverse to the long axis of the bone plate (or of the plate portion that defines this aperture). The toothed aperture may be disposed in only one plate portion, such as in an external or internal plate portion, or may extend to be included in two or more plate portions, for example, by extending across a bridge portion of the bone plate. In some examples, each of two or more plate portions of the bone plate may include a distinct toothed aperture. In some examples, one plate portion may have two or more toothed apertures. The toothed aperture may be configured to receive and axially retain one fastener or two or more fasteners.

The toothed aperture may have any suitable number and arrangement of projections. The toothed aperture may have a single projection or may have a set of two or more projections. At least a subset of the projections may be arrayed along the perimeter of the aperture to form an array of projections or teeth. The term “along,” as used herein relative to an aperture, means arranged generally parallel to the long axis of the aperture. The array of projections may be a substantially linear array, generally with adjacent pairs of projections of the array separated by a depression (such as a groove). The linear array may be described as an array of thread-like segments, although each segment may be nonhelical and/or defined by a generally noncylindrical surface (in contrast to a standard thread or thread segment). The toothed aperture may have all of its projections disposed on the same side of the aperture or may have a subset of one or more projections disposed on each side of a pair of opposing sides (wall portions) of the aperture. In some examples, the toothed aperture may have opposing subsets of projections disposed on opposing sides of the aperture. The opposing subsets may have the same (or a different) spacing of projections. Corresponding, opposing projections of the opposing subsets may be aligned transversely or may be offset, for example, offset along the aperture by about one-half or one-fourth the spacing, among others, between adjacent pairs of projections.

Adjacent projections may have any suitable spacing. For example, the projections may be spaced corresponding to the pitch of the thread(s) of a threaded fastener (or an integral multiple of the pitch). (In some examples, the pitch may correspond to an integral multiple of the spacing of the projections.) A spacing corresponding to the pitch may restrict the angular disposition (or range of angular dispositions) at which the fastener can be engaged with the toothed aperture. In some examples, the spacing may be adjusted to allow a different angle of approach of the fastener (such as from different openings) and/or to permit a fastener to engage the projections from a set of two or more angular dispositions (such as different orientations of the fastener from the same opening). The spacing between the projections may be the same along the aperture. Alternatively, the spacing may change along the aperture so that, for example, threaded shanks, with threads of different pitch, may be received at different positions along the toothed aperture.

The projections may have any suitable structure. The projections may be configured, for example, as ridges, knobs, or prongs, among others. The ridges may have crests that are blunt or sharp. The ridges may have flanking sides defined by flat, convex, or concave surfaces, among others.

The ridges, and particularly their crests, may extend at any suitable orientation (and thus angle) relative to the long axis of the aperture and/or relative to a length-by-width plane defined by the plate portion in which the ridges are disposed. For example, the ridges may be oblique ridges that extend obliquely to this plane. The orientation of the ridges may determine, at least partially, the orientation and/or range of angles at which a fastener can be received by the toothed aperture. For example, ridges oriented more nearly orthogonal to the plane of the toothed aperture (or plate portion), that is, oriented at a larger angle relative to this plane, may receive and retain a fastener approaching from a smaller angle to the toothed aperture, that is, shallower or more nearly tangential to the aperture (for example, from an opening disposed closer to the toothed aperture). Similarly, ridges oriented more nearly parallel to the plane of the toothed aperture (or plate portion), that is, oriented at a smaller angle relative to this plane, may receive and retain a fastener approaching from a larger angle to the toothed aperture, that is steeper or more nearly orthogonal to the aperture (for example, from an opening disposed farther from the toothed aperture). Accordingly, the orientations/angles of the ridges may vary along the length of the aperture to accommodate the approach of a fastener from different orientations and/or different openings (see Example 4 below). A fastener disposed through a given opening in a first portion of a plate commonly would approach farther (more distal) regions of a toothed aperture in a second portion of the plate at a shallower or more nearly tangential angle than it would approach nearer regions of the toothed aperture. Therefore, ridges in farther regions of a toothed aperture may be oriented at larger angles relative to the plane of the toothed aperture, ridges in nearer regions of the toothed aperture may be oriented at smaller angles relative to the plane of the toothed aperture, and ridges in intermediate portions of the tooth aperture may be oriented at angles that vary continuously and/or discontinuously between these two extremes. Consequently, the average ridge-to-ridge spacing (or the number of ridges per unit length) may be smaller toward the proximal end of the toothed aperture (relatively closer to the bridge portion of the plate) and larger toward the distal end of the toothed aperture (relatively farther from the bridge portion). Similarly, the length of these ridges may be relatively smaller toward the proximal end of a toothed aperture, and relatively larger toward the distal end of the toothed aperture.

The orientations/angles of the ridges may be the same or different on opposing sides of the aperture. In some examples, the orientation of the ridges may be approximately orthogonal to an axis defined by an opening that directs a fastener along the axis to the toothed aperture. In some examples, different orientations of the ridges on opposing sides of the aperture may, for example, be suitable to more accurately match a thread of a fastener, locally on opposing sides of the fastener's shank. For example, the ridges on one side of the aperture may define an obtuse angle with the axis and ridges on the other side of the aperture may define an acute angle with the axis, in correspondence with opposing sides of a fastener's helical thread (see Example 4 below).

The projections may have any suitable length and height. For example, the projections may be formed as longer ridges (measured along their crests) to decrease the permitted range of angular approach of a fastener to the aperture or as shorter ridges to increase this permitted range. The length of the ridges may be defined at least substantially by the local thickness of the plate and the orientation of the ridges (for ridges that extend between opposing surfaces of the plate) and/or may be shorter (or longer) than this local thickness. The height of projections (measured as the distance from the crest to the base of a projection) may correspond at least substantially to the height of a thread on a fastener (measured from the crest to the root of the thread). Alternatively, the height of projections may be less or more than the height of the thread on the fastener.

Depressions disposed between the projections may have any suitable structure, generally as defined by the structure of flanking projections. For example, narrower projections having concave sides may define wider depressions, and wider projections having convex sides may define narrower depressions, everything else being equal. Accordingly, the shape of the projections and depressions may determine, at least partially, the range of angles at which a fastener can be engaged with the toothed aperture and/or how tightly the fastener fits into the toothed aperture. In some examples, the toothed aperture may be configured so that a received fastener is tightly engaged. Alternatively, the toothed aperture may be configured so that a received fastener can be jiggled axially (in the absence of bone).

The toothed aperture may have any suitable width. The ridges may have a constant width or, for example, may taper toward one or both opposing ends. Accordingly, portions of the toothed aperture may flare toward a fastener-entry side of the toothed aperture, to facilitate guiding a fastener into the aperture.

IV. Methods of Using Bone Plates with Toothed Apertures

Bone plates having toothed apertures, as described herein, may be attached to or otherwise associated with bone using any suitable method. In an exemplary method, a practitioner may perform any suitable combination of the following steps in any suitable order: (1) select a target bone(s) having a discontinuity; (2) select a suitable bone plate having a toothed aperture; (3) select one or more suitable fasteners for use with the bone plate and the target bone, including a threaded fastener configured to be received in the toothed aperture; (4) prepare the target bone(s) for installation of the bone plate (for example, position bone fragments (such as to reduce a fracture) and/or remove cartilage (such as for bone fusion at a joint); (5) adjust the shape of the plate, if desired, to adjust the fit of the bone plate in relation to the target bone(s); (6) place a first (internal) portion of the bone plate in the target bone (such as by insertion into a preformed hole in the bone and/or by forcing the internal portion into the bone); (7) secure a second (external) portion of the bone plate to a surface of the target bone using one or more of the fasteners selected; and (8) place one or more fasteners through one or more openings of the external portion of the plate, through an adjacent bone fragment and across the discontinuity into a spaced bone fragment, and into threaded engagement with the toothed aperture of the internal portion. Step (8) may be facilitated, for example, by using an imaging apparatus (such as a fluoroscope) and/or an external guide device to guide placement of the fastener. Exemplary external guide devices are described in the following patent application, which is incorporated herein by reference: U.S. patent application Ser. No. 10/717,401, filed Nov. 19, 2003.

The bone plates described herein may be fabricated by any suitable methods. The bone plates or features thereof may be cast, machined, drilled, stamped, formed, and/or bent, among others.

The bone plates described herein may be provided as kits. The kits may include one or more bone plates having a toothed aperture. One or more fasteners configured to be received in the toothed aperture, particularly from an opening in another portion of the plate.

### V. EXAMPLES

The following examples describe selected aspects and embodiments of the present teachings, including exemplary bone plates with toothed apertures and methods of using these exemplary bone plates to fix bones. These examples and the various features and aspects thereof are included for illustration and are not intended to define or limit the entire scope of the present teachings.

### Example 1

### Exemplary Bone Plate with a Toothed Aperture

This example describes an exemplary bone plate including a toothed aperture, in accordance with aspects of the present teachings; see FIGS. 2-7.

FIG. 2 shows bone plate 22 in the absence of fasteners and bone; bone plate 22 may be used as a blade plate. Various features of this bone plate, such as toothed aperture 24, external portion 34, internal portion 36, and opening 44 were introduced above in relation to FIG. 1.

External and internal portions 34, 36 may include a plurality of openings. For example, the external portion may include a first set of one or more openings 50, a second set of one or more openings 52, and an oblique opening 44 disposed between the first and second sets. The internal portion 36 may include toothed aperture 24 and one or more additional openings. A bridge portion 54 may include one or more bridge openings 56 disposed generally at the junction between the external and internal plate portions. Each opening may be threaded or nonthreaded and may include (or lack) a counterbore 58 configured, for example, to reduce protrusion of a fastener's head above the outer surface of the plate.

Bridge portion 54 may connect the exterior and interior portions of bone plate 22. The bridge portion may be configured to span a bone discontinuity. Alternatively, or in addition, the bridge portion may define, at least partially, the angular disposition of the external and internal portions of the bone plate. For example, here, the bridge portion includes an approximate right-angle bend so that the plate portions extend transverse to one another. However, the bridge portion may define any suitable angle and may be configured to permit pre- and/or peri-operative adjustment of the shape of the plate. The bridge portion thus may be narrowed, thinned, annealed, and/or otherwise configured and/or treated to facilitate bending at this portion. Bridge openings (such as opening 56) may permit bending and/or may be suitable to direct fasteners at oblique angles relative to one or more of the plate portions, among others.

FIG. 3 shows bone plate 22 holding a bone screw 42 in threaded engagement with toothed aperture 24. The bone screw may include a head 60 and a shank 62. Head 60 may be nonthreaded (as shown here) or may include a thread configured to lock the head to the plate. The shank may be a threaded shank that includes a distal threaded region 64 and a proximal nonthreaded region 66. Alternatively, the bone screw may include a thread(s) that extends substantially or completely along the shank. The thread may be a relatively deep thread, such as the thread found in cancellous bone screws, or may be a relatively shallow thread, such as the thread found in cortical bone screws.

Screw 42 may span external and internal plate portion 34, 36 and lock to toothed aperture 24, to provide intra-plate tension between the plate portions. For example, the screw may be received first in oblique opening 44 and then thread into toothed aperture by turning the bone screw so that threaded region 64 advances into and engages the projections of toothed aperture 24. With the head of the screw engaged with the external plate portion, further rotation of screw 42 and thus further advancement of threaded region 64 into/through the aperture applies a tension to the plate.

The toothed aperture, bone screw, and oblique opening may be configured to permit the bone screw to thread into the toothed aperture along two or more distinct axes. In the present illustration, bone screw 42 may threadably engage the toothed aperture along axis 68. Alternatively, bone screw 42 may threadably engage the toothed aperture along alternative axis 70, as illustrated with the bone screw shown at 72 in phantom outline. Accordingly, a surgeon has greater flexibility in selecting a path for intra-plate placement of the bone screw.

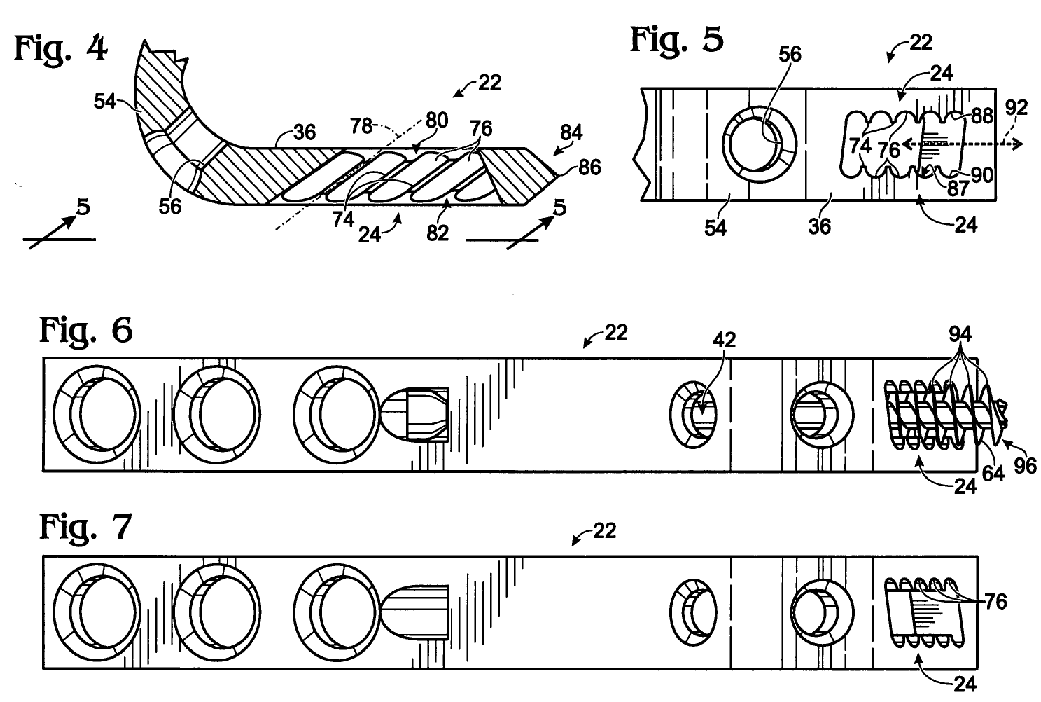

FIG. 4 shows selected portions of bone plate 22, particularly bridge portion 54 and internal portion 36. Locking aperture 24 may include a plurality of projections (ridges 74) and depressions (grooves 76). The ridges and grooves may extend obliquely, shown at 78, relative to the aperture. Ridges are considered as “oblique ridges” when they extend obliquely to the long axis of the toothed aperture. Furthermore, the toothed aperture may widen or flare toward the external plate portion, shown at 80, and/or opposing the external plate portion, shown at 82.

Internal portion 36 may be configured to be placed into bone. Accordingly, the interior portion may include a leading end region 84 that is beveled or tapered, to facilitate introducing the interior portion into the bone. In some examples, the leading end region may include a relatively sharp leading edge 86.

FIG. 5 shows a view of toothed aperture 24 taken generally parallel to the orientation of ridges 74 and grooves 76. The ridges may have concave sides and a blunt crest. The grooves may be cylindrical, that is, corresponding to a portion of a cylinder (such as a half-cylinder).

The toothed aperture may be defined by a wall 87 of the internal plate portion. The wall may be distinct from inner and outer surfaces of the bone plate and/or may be included in one or both of these surfaces. The wall may include opposing wall portions 88, 90, which may extend along the perimeter of the aperture in a generally parallel relationship, as shown here. The ridges and/or grooves thus may be disposed along each wall portion, that is, arrayed generally parallel to the long axis 92 of the toothed aperture. In some examples, the ridges/grooves (and/or projections/depressions) may be disposed in a linear array along each wall portion. Each ridge and groove (and/or projection and depression) may be defined by, and thus restricted to, only one of the opposing wall portions. One or more (or all) projections of one opposing wall portion may be offset along the long axis of the aperture relative to one or more (or all) projections of the other opposing wall portion. The offset may be a fraction of the spacing between adjacent projections, such as about one-half or about one-fourth of this spacing.

FIGS. 6 and 7 show bone plate 22 viewed generally along line 6-6 of FIG. 3, in the presence and absence, respectively, of bone screw 42. When disposed in threaded engagement with toothed aperture 24, individual thread segments 94 of the bone screw (FIG. 6) may be received in individual grooves 76 of the toothed aperture (see FIGS. 6 and 7). In some examples, the bone screw may be advanced until a distal section of threaded portion 64 has traveled through the toothed aperture, to be disposed distally thereof, shown at 96.

In an exemplary embodiment, intended only for illustration, bone plate 22 may have the following dimensions: overall unbent length—4.667 inches; bent length—3.542 inches; length from bridge portion opening to end of external portion—3.407 inches; length from bridge portion opening to end of internal portion—1.260 inches; width of plate 0.433 inches; thickness of plate 0.189 inches; angle of axis defined by the oblique opening in relation to the long axis of the internal portion—66 degrees; and spacing between ridges of the toothed aperture (measured parallel to the long axis of the internal portion)—0.146 inches.

### Example 2

### Exemplary System for Guiding a Fastener to a Toothed Aperture

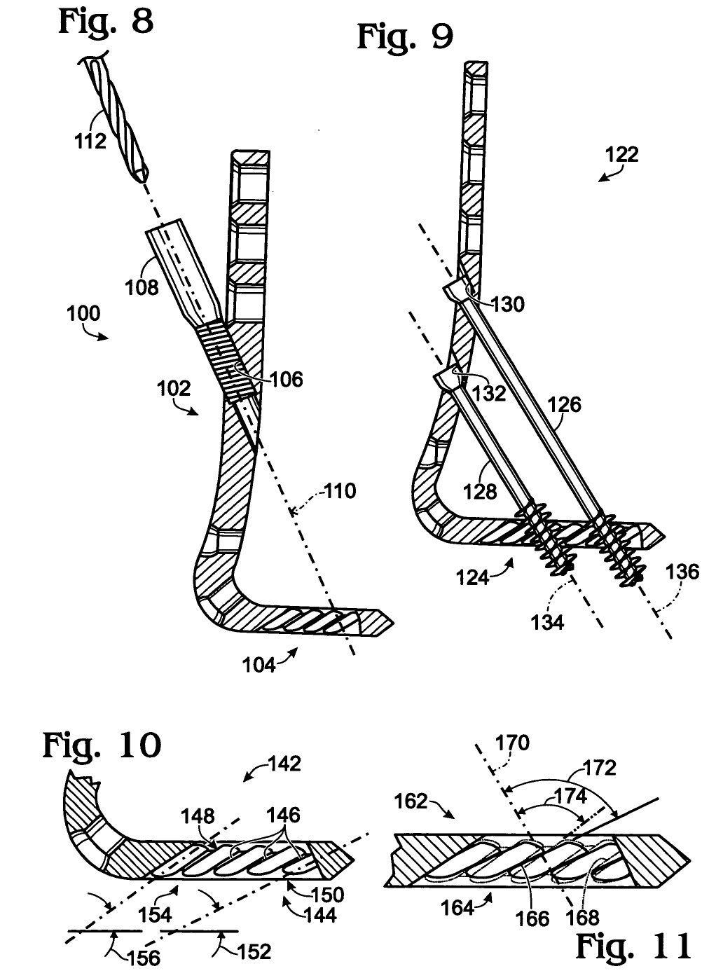

This example describes an exemplary system for defining a guide axis that extends through a toothed aperture; see FIG. 8.

FIG. 8 shows a system 100 for drilling and fastener placement. The system includes another exemplary bone plate 102 with a toothed aperture 104. The bone plate include a threaded opening 106 configured to threadably receive a cannulated drill guide 108. The drill guide, when engaged with the bone plate in threaded opening 106, may define an axis 110 along which a fastener may threadably engage the toothed aperture. Accordingly, a drill 112 may be guided along the drill guide to form a hole centered about guide axis 110. After hole formation in bone, a bone screw or other suitable fastener then may be placed into the threaded aperture and rotatably engaged with toothed aperture 104. The bone screw may be guided to the toothed aperture while the drill guide is still engaged with the plate and/or after the drill guide is removed. The bone screw thus may have a threaded or nonthreaded head for locking or nonlocking engagement, respectively, threaded opening 106.

In other embodiments, a drill guide may be employed that does not threadably engage the bone plate. In these embodiments, the axis defined by the drill guide may be adjustably selected by a surgeon, to permit greater flexibility in selection of an axis along which a threaded fastener engages the toothed aperture.

### Example 3

### Exemplary Bone Plate with Increased Intra-Plate Coupling

This example describes an exemplary bone plate with a locking aperture targeted by a plurality of plate openings; see FIG. 9.

FIG. 9 shows another exemplary bone plate 122 with a toothed aperture 124. Bone plate 122 is configured so that a plurality of threaded fasteners 126, 128 may be received in a plurality of oblique openings 130, 132 and extend to toothed aperture 124 for threaded engagement therein. The fasteners may extend to the toothed aperture along parallel (or nonparallel) axes 134, 136.

### Example 4

### Exemplary Bone Plates with Nonparallel Ridges

This example describes exemplary bone plates having toothed apertures with nonparallel ridges; see FIGS. 10 and 11.

FIG. 10 shows the internal and bridge portions of an exemplary bone plate 142 having a toothed aperture 144 with nonparallel ridges 146. The nonparallel ridges are defined by one of the opposing wall portions 148 of the plate. (In other words, these ridges are on the same side of the toothed aperture.) Ridges closer to the leading end of the internal portion, shown at 150, may extend at a smaller angle(s) (such as angle 152) relative to the long axis of the internal portion (more nearly parallel to this long axis). Ridges closer to the trailing end of the internal portion, shown at 154, may extend at a larger oblique angle(s) (such as angle 156) relative to the long axis of the toothed aperture (more nearly perpendicular to this long axis).

FIG. 11 shows the internal portion of an exemplary bone plate 162 having a toothed aperture 164 with nonparallel ridges 166, 168. Ridges 166 disposed on the far side of the aperture are drawn in solid lines, as in a standard sectional view. (The “far side” corresponds to the wall portion on the left side of the toothed aperture when viewed from the external portion of the plate along fastener placement axis 170.) Ridges 168 disposed on the opposing, near side (wall portion on the right side) of the aperture are drawn in phantom outline because they normally would not be visible in this sectional view. Here, ridges 166 of the left-sided wall portion define an obtuse angle 172 with axis 170, and ridges 168 of the right-sided wall portion define an acute angle 174 with axis 170. These obtuse and acute angles may be selected so that the ridges on the opposing sides of the toothed aperture more closely correspond to the local thread angle on opposing sides of the fastener's threaded shank.

### Example 5

### Selected Embodiments

This example describes selected embodiments of the invention, presented as a series of indexed paragraphs.

1. A bone plate, comprising: (A) a first portion defining an opening; and (B) a second portion connected to the first portion and having a wall defining an aperture, the wall including at least one projection configured to retain a fastener placed through the opening and advanced rotationally into the aperture.

2. The bone plate of paragraph 1, wherein the opening is elongate.

3. The bone plate of paragraph 1, wherein the opening is circular and defines an axis extending obliquely to the long axis of the first portion.

4. The bone plate of paragraph 1, wherein the first portion includes a bone-facing surface that is substantially convex along a path extending generally parallel to the long axis of the first portion.

5. The bone plate of paragraph 1, wherein the first portion defines a plurality of elongate openings.

6. The bone plate of paragraph 1, wherein the first portion defines a plurality of openings, and wherein the at least one projection is configured to retain, concurrently, at least two fasteners placed through at least two of the plurality of openings and advanced rotationally into the aperture.

7. The bone plate of paragraph 1, wherein the at least one projection is a set of projections.

8. The bone plate of paragraph 7, wherein the set of projections includes a linear array three or more projections.

9. The bone plate of paragraph 8, wherein the spacing between each adjacent pair of projections of the linear array is at least substantially the same.

10. The bone plate of paragraph 7, wherein the wall includes a pair of opposing walls, and wherein the set of projections includes one or more projections included in each of the opposing walls.

11. The bone plate of paragraph 10, wherein each of the opposing walls includes three or more projections.

12. The bone plate of paragraph 11, wherein the three or more projections of each opposing wall are disposed in a linear array.

13. The bone plate of paragraph 10, wherein each of the opposing walls includes at least two projections having a spacing between the at least two projections, and wherein the spacing of the at least two projections is at least substantially the same for each of the opposing walls.

14. The bone plate of paragraph 10, wherein one or more projections of one of the opposing walls is offset from the at least one projection of the other opposing side.

15. The bone plate of paragraph 14, wherein one of the opposing walls includes two or more projections having a spacing, the at least one projection of the other opposing wall being offset by one-half the spacing in relation to a transverse axis defined by the toothed aperture.

16. The bone plate of paragraph 1, wherein the at least one projection is a plurality of projections and the openings are a plurality of openings, and wherein the toothed aperture is configured to retain at least two fasteners placed through at least two of the plurality of openings and advanced rotationally into the aperture.

17. The bone plate of paragraph 1, wherein the aperture defines a plane, and the at least one projection has a crest that extends obliquely to the plane.

18. The bone plate of paragraph 17, wherein the at least projection includes a plurality of projections disposed in a linear array, and wherein the crests of the plurality of projections are at least substantially parallel.

19. The bone plate of paragraph 1, wherein the at least one projection includes a set of projections separated by depressions, and wherein the projections have sharp crests and wherein the depressions have rounded bottoms.

20. The bone plate of paragraph 1, wherein the first portion is configured to appose an exterior surface of the bone, and wherein the second portion is configured to be placed in the bone.

21. A bone plate comprising a wall defining an elongate aperture and including opposing projections configured to retain a fastener advanced rotationally into the aperture.

22. The bone plate of claim 21, wherein the opposing projections including opposing arrays of projections.

23. The bone plate of claim 22, wherein each opposing array of projections includes a set of ridges.

24. The bone plate of claim 23, wherein the ridges of the set extend at least substantially parallel to one another.

25. The bone plate of claim 23, wherein the elongate aperture defines a plane, and wherein the ridges extend obliquely to the plane.

26. A method of securing the bone plate of any of the preceding numbered paragraphs to a bone, comprising: (A) selecting the bone plate of any one of paragraphs 1-25; and (B) advancing a fastener rotationally into the elongate aperture so that the at least one projection retains the fastener in the elongate aperture.

27. A system or kit for repairing bones, comprising (A) a bone plate having a wall defining an aperture, the wall including at least one projection; and (B) a bone fastener configured to be placed into the aperture and retained therein by the at least one projection.

28. The system or kit of paragraph 27, wherein the at least one projection includes a pair of opposing linear arrays, each opposing linear array including two or more projections.

29. The system or kit of paragraph 28, wherein the two or more projections are configured as ridges having an orientation, and wherein the orientation defines a permissible range of angles from which the bone fastener can be placed into the aperture.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

1. A bone plate for bone fixation, comprising:
a plate member configured to engage and support a bone and including a wall defining an aperture, the wall including a plurality of teeth disposed along the aperture.

2. The bone plate of claim 1, wherein the wall includes a pair of opposing wall portions disposed in a generally parallel relationship, and wherein each of the plurality of teeth is restricted to one of the wall portions.

3. The bone plate of claim 2, wherein one or more teeth of one opposing wall portion are offset along the aperture from one or more teeth of the other opposing wall portion.

4. The bone plate of claim 1, wherein the plurality of teeth includes a plurality of oblique ridges.

5. The bone plate of claim 1, wherein the plurality of teeth includes a substantially linear array of at least three projections disposed along the aperture.

6. The bone plate of claim 1, wherein the aperture includes opposing ends, and wherein the plurality of teeth is spaced from the opposing ends.

7. The bone plate of claim 1, wherein the plate member includes a first plate portion defining an opening and a second plate portion defining the aperture, and wherein the opening is disposed so that a threaded fastener can be received in at least the opening and advanced rotationally into threaded engagement with one or more of the plurality of teeth, thereby spanning the first and second plate portions with the threaded fastener.

8. The bone plate of claim 7, wherein the first plate portion is configured to be placed on bone, and wherein the second plate portion is configured to extend into bone.

9. The bone plate of claim 8, wherein the second plate portion includes a leading end region, and wherein the leading end region is tapered.

10. A system for bone fixation, comprising:
a fastener; and
a bone plate including a first plate portion defining an opening and a second plate portion including a wall defining an aperture, the wall including a plurality of projections configured to retain the fastener extending along either of at least two axes from the opening.

11. The system of claim 10, wherein the fastener includes a head and a shank, and wherein the shank includes a nonthreaded region adjacent the head and a threaded region spaced from the head.

12. The system of claim 10, wherein the at least two axes are nonparallel.

13. The system of claim 10, wherein the second plate portion defines a long axis, and wherein the plurality of projections includes oblique ridges extending at one or more oblique angles relative to the second plate portion.

14. The system of claim 10, wherein the bone plate is generally L-shaped.

15. The system of claim 10, wherein the first plate portion is configured to be disposed on bone, and wherein the second plate portion is configured to be disposed in bone.

16. A method of fixing bone, comprising:
selecting a bone plate including a first plate portion defining an opening and a second plate portion including a wall, the wall defining an aperture and including a plurality of projections disposed along the aperture;
disposing the first plate portion on bone and the second plate portion in bone;
placing a fastener through the opening and into the aperture so that at least one of the plurality of projections engages the fastener to restrict axial movement of the fastener.

17. The method of claim 16, wherein the step of disposing is performed at least partially on a tibia bone.

18. The method of claim 16, wherein the step of placing includes a step of selecting an axis for fastener placement from a set of two or more nonparallel axes along which the fastener can engage one or more of the plurality of projections.

19. The method of claim 16, wherein the aperture includes opposing ends, and wherein the step of placing disposes the threaded fastener in a spaced relationship with each of the opposing ends.

20. The method of claim 16, further comprising a step of forming a hole in bone along an axis extending through the opening and the aperture before the step of placing.
